(19) United States
(12) Patent Application Publication
(10) Pub. No.: US 2005/0245890 A1
(43) Pub. Date: Nov. 3, 2005

(54) Device and method for delivering biological agents to the respiratory system of test animals

(76) Inventor: David L. Jackson, Yadkinville, NC (US)

Correspondence Address: CAHN & SAMUELS LLP, 2000 P STREET NW, SUITE 200, WASHINGTON, DC, 20036, US

(21) Appl. No.: 11/079,484

(22) Filed: Mar. 15, 2005

Related U.S. Application Data
(60) Provisional application No. 60/552,713, filed on Mar. 15, 2004.

(51) Int. Cl. A61M 31/00

(52) U.S. Cl. 604/500

(57) ABSTRACT

A method of delivering a biological agent to a laboratory animal includes sedating the animal and inserting a conduit into the animal's mouth after it has been sedated and positioning the conduit such that a distal end of the conduit is proximate to a lower lobe of the animal's lung. A biological agent is then injected into the conduit. A propellant is injected into the conduit to help direct the biological agent to the animal's lungs. A flush is injected into the conduit and the conduit is removed from the animal's mouth.

This application claims priority from provisional application Ser. No. 60/552,713 filed Mar. 15, 2005, and is herein incorporated by reference in its entirety.

### I. FIELD OF THE INVENTION

This invention relates to biological agent delivery systems, methods and devices for primates. More particularly, this invention relates to devices and methods for delivering biological agents, e.g., viruses, bacterium, etc., to the respiratory system of test animals.

### II. BACKGROUND OF THE INVENTION

In conducting infectious disease research, it is often useful to study the effects of viruses and bacterium on laboratory animals. In order to conduct such studies, researchers must first infect the animals with the virus or bacterium that they wish to study.

Heretofore, researchers have infected lab animals by several methods including IV injection, exposure through an inhalation chamber, and by depositing a virus or bacterium suspension onto the mucous membrane of the mouth or into the eye. These methods have proven to be less than ideal.

With respect to the IV injection method, in a particular known method a primate is given an intramuscular (im) injection of a sedative, e.g., Telazol.® Once the primate is sedated, a vein is located in the lower limb. Hair is then removed from the target area and an injection site is cleaned. One problem with this method is that sometimes the veins are too small in the selected site so it is necessary to prepare a second site. In some instances it may be necessary to inject the femoral artery or a vein in the arm. During the process of searching for the optimal site, the animal may arise, necessitating another dose of sedative. Also, there is always a risk that the researcher may miss the vein and accidentally inject itself. Likewise the researcher may miss the vein or put the biological agent in other tissues which may cause sluffing of surrounding tissue and destruction of the vein. There is also the possibility of a needle prick while holding the vein to guide the injection needle in. Of course such a prick may cause severe illness or even death to the researcher.

Another problem with the injection method is that it does not mimic the procedure in which viruses and bacterium are normally introduced to the system. The first outbreak will typically occur at the point of injection. Accordingly, it may be difficult to determine, e.g., how a virus or bacteria normally travels through the body.

Still another problem with the injection method is that, with certain animals, it may take up to 72 hours for them to become infected. During that entire incubation period, the researcher is observing the animal and wearing a protective suit. If, as is common, multiple animals are being infected for a study, e.g., 10-15, the researcher may remain in the protective suit for 8-10 hours or longer.

Inhalation chambers present their own set of problems. Animals that are introduced to biological agents via inhalant chambers must be manually cleaned by researchers. Although the researchers employ bio safety hoods, in the cleaning process there is an unnecessary risk of direct exposure to the biological agent through contact and/or inhalation. Also, an irritable animal having just been confined to a small chamber and subject to a caustic biological agent may bite, and thereby infect, the researcher.

An additional problem with inhalant chambers is that it is very difficult to determine the amount of biological agent that the animal has ingested. One cannot reliably determine how much of the biological agent went into the eye, the nose or the lungs. Also it is difficult to determine the duration of exposure necessary for infection and the amount of biological agent necessary for infection.

With respect to direct deposit of the biological agent, whether onto the mucous membrane of the mouth or in the eye, this is a particularly difficult procedure for the researcher to perform. It requires a great deal of skill and precision on the researchers part and still presents an unnecessary risk of contamination to the researcher due to spills.

In view of the foregoing, there is a need for a device and method for delivering biological agents to test animals that minimizes the risk of researcher exposure and contamination.

### III. SUMMARY OF THE INVENTION

A biological agent delivery device is provided in accordance with an embodiment of the invention. The delivery device includes an outer tube having a proximal end and a distal end. An inner tub is disposed within said outer tube having a proximal end and a distal end, the distal end extends beyond the distal end of said outer tube. A connector is coupled to the proximal end of said outer tube. A a multi-port stop cock having a first port, a second port and a third port, is provided, the first port is connected to the proximal end of the inner tube. A biological agent reservoir is connected to the second port of the stop cock and a a propellant delivery device is connected to the third port of said stop cock. The delivery device further includes a mouth guard sealingly coupled to a downstream portion of said connector.

Given the following enabling description of the drawings, the apparatus should become evident to a person of ordinary skill in the art.

### IV. BRIEF DESCRIPTION OF THE DRAWINGS

FIG. 1 illustrates depicts a biological agent delivery device according to a first embodiment of the invention.

FIG. 2 depicts a biological agent delivery device according to a second embodiment of the invention.

FIG. 3 illustrates a flow chart showing the method of infecting a test animal in accordance with an embodiment of the invention.

FIG. 4 illustrates positioning of the delivery device in the animals lungs in accordance with the invention.

### V. DETAILED DESCRIPTION OF THE DRAWINGS

The present invention is directed to methods and devices for delivering biological agents to laboratory animals. As used herein, the term biological agent includes but is not limited to viruses and bacterium. In accordance with the invention, the laboratory animal is preferably sedated and a tube is inserted through the animal's mouth preferably to the lower lobe of the animal's lung. The tube forms a biological agent delivery path. The biological agent is fed through the tube and the tube is flushed with a rinsing agent. By this method, the animal may be safely infected with the biological agent within 24 hours or less.

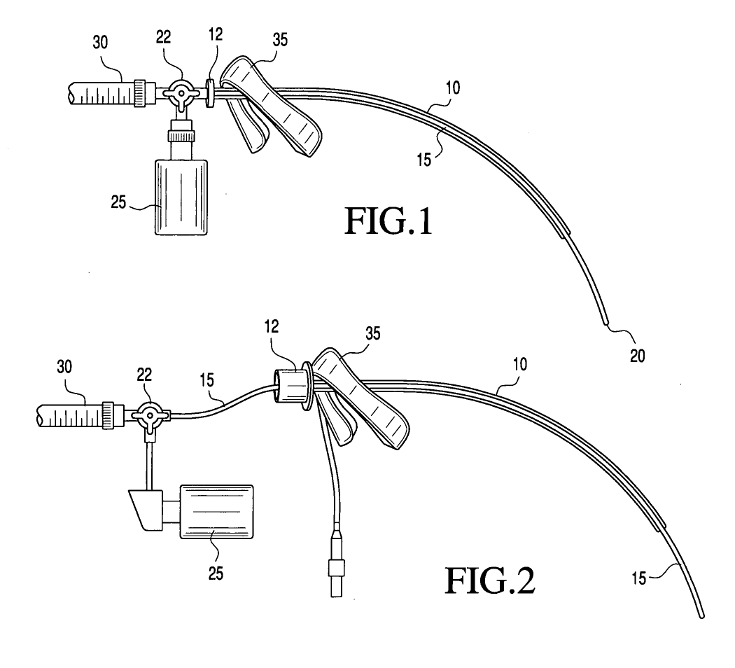

FIG. 1 illustrates a delivery device according to an embodiment of the invention. The delivery device includes an outer tube 10 having a connector 12 disposed on a proximal end and having a substantially open distal end. Outer tube 10 is preferably between about 20 cm to 40 cm or longer and preferably has an inner diameter of between about 2 cm to about 6 cm. In accordance with one embodiment of the invention, outer tube 10 is a standard 4.0 endotracheal tube.

Disposed within outer tube 10 is inner tube 15. Inner tube 15 preferably of sufficient length to extend from the mouth of the laboratory animal as far as the lower lobe of its lungs. Accordingly, the preferred length of inner tube 15 will vary depending upon the type of laboratory animal for which it is used. However in some embodiments, inner tube 15 may be between about 25 cm to about 60 cm or longer.

Inner tube 15 preferably includes a distal end 20 that extends through the distal end of outer tube 10. Distal end 20 is preferably rounded so as not to damage any lung tissue with which it may come into contact. An exemplary inner tube may be a conventional premature infant feeding tube having an inner diameter of about 1.7 cm and having a length of about 38 cm.

The proximal end of inner tube 15 is preferably attached to a multi-port connector 22. Also attached to multi-port connector 22 is a propellant delivery device 25 and a syringe 30 or equivalent structure for delivering the biological agent into inner tube 15. In preferred embodiments, multi-port connector 22 is preferably a stop cock, e.g., a 3 way stop cock. In accordance with one embodiment, multi-port connector 22 is directly coupled to connector 12 as illustrated in FIG. 1. In other embodiments, it may be desirable to have separation between syringe 30, propellant delivery device 25 and the animal's mouth. In such an embodiment, inner tube 15 may extend through the proximal end of outer tube 10 and be connected to multi-port connector 22 upstream from the proximal end of outer tube 10, as illustrated in FIG. 2.

To minimize leakage of biological agent through the animal's mouth, a mouth guard 35 is coupled to connector 12 as illustrated in FIG. 2. Mouth guard 35 is preferably designed to fit over the mouth of the animal being infected and is preferably further provided with a filter to trap any biological agent that may come back up through inner tube 15 or even outer tube 10.

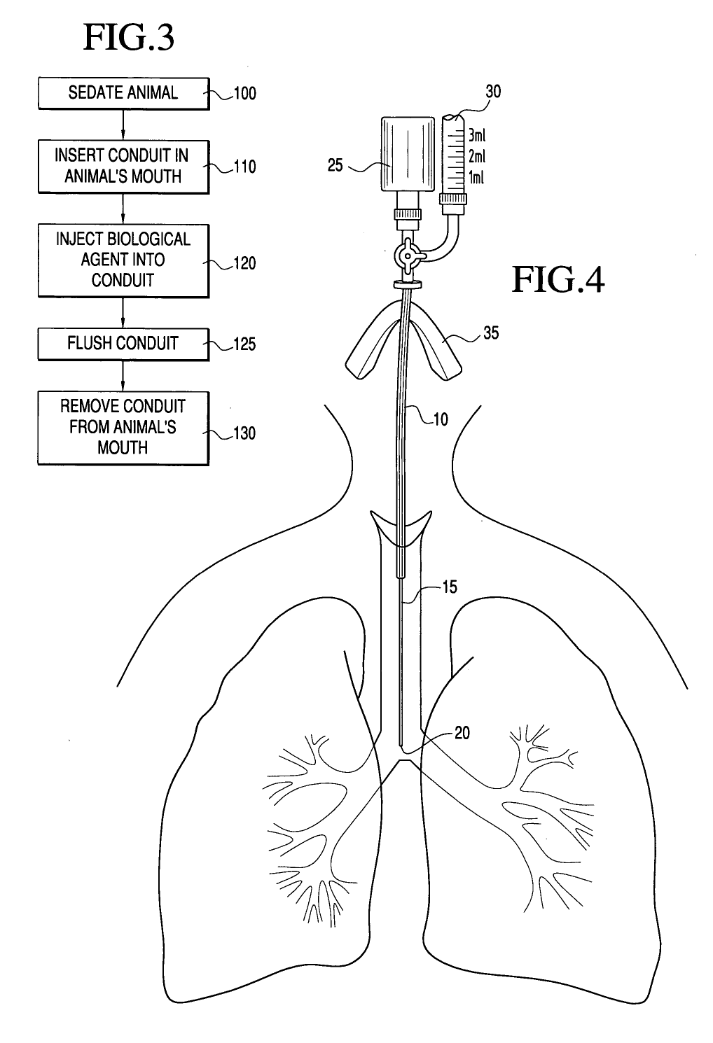

Turning to FIG. 3, biological agent may be delivered to the animal using the delivery device of the invention according to the following procedure. In step 100, the animal is sedated. The animal is preferably given a sedative in an appropriate dosage to rapidly put the animal to sleep. The appropriate dosage will of course depend upon the animal being infected. A commonly used sedative is Telazol®.

Once the animal has fallen asleep, in step 110, a conduit is inserted in the animal's mouth. In preferred embodiments, the conduit includes outer tube 10 and inner tube 15. The conduit is preferably inserted such that distal end 20 of inner tube 15 is disposed proximate to the lower lobe of the animal's lung as illustrated in FIG. 4.

Once the conduit is in place, in step 120, the biological agent is delivered to the animal's lung. Delivering the biological agent directly to the lower lobe of the lung is believed to cause rapid infection, i.e., within 24 hours or less. Delivering the biological agent includes directing the biological agent from syringe 30 by opening the port on the stop cock to which syringe 30 is connected and activating the propellant delivery device 25 to direct propellant into inner tube 15 to drive biological agent downstream to the lungs. Preferably syringe 30 is emptied before propellant is directed into inner tube 15. A preferred propellant delivery device is an air canister. However, other propellant delivery devices may be employed within the scope of the invention.

After syringe 30 is emptied, in step 125, inner tube 15 is preferably flushed by either replacing syringe 30 with a second syringe containing the flush or by adding flush to syringe 30. A preferred flush is a common phosphate-buffered sucrose (pbs) solution. Inner tube 15 maybe further flushed by directing propellant through the tube as in step 120 to drive the solution into the animal's lungs.

In step 130, the conduit is removed from the animal's mouth.

The present invention provides several advantages of conventional biological agent delivery methods and devices. Biological agent loads and flush may be drawn prior to entering the laboratory thus eliminating the worry of accidental needle pricks and possible self-inoculation. A technician can intubate the animal and administer the biological agent thus freeing the researcher to perform other tasks or the researcher may perform all tasks himself with a minimum amount of stress. Animal infection can often be achieved in 24 hours or less. Biological agent delivery in accordance with the invention closely mimics the natural method of infection which allows for more accurate study.

Those skilled in the art will appreciate that various adaptations and modifications of the above-described preferred embodiments can be configured without departing from the scope and spirit of the invention. For example, although the above described embodiments disclose concentric tubes 10 and 15, the present invention could be implemented with single tube. Therefore, it is to be understood that, within the scope of the appended claims, the invention may be practiced other than as specifically described herein.

1. A biological agent delivery device comprising:
an outer tube having a proximal end and a distal end;
an innter tube disposed within said outer tube having a proximal end and a distal end, the distal end extending beyond the distal end of said inner tube;
a connector coupled to the proximal end of said outer tube;
a multi-port stop cock having a first port, a second port and a third port, the first port being connected to the proximal end of said inner tube;
a biological agent reservoir connected to the second port of the stop cock;
a propellant delivery device connected to the third port of said stop cock; and
a mouth guard sealingly coupled to a downstream portion of said connector.

2. A method for delivering a biological agent to a laboratory animal comprising:
sedating the laboratory animal;
inserting a conduit into the animal's mouth and positioning the conduit such that a distal end of the conduit is proximate to a lower lobe of the animal's lung;
injecting the biological agent into the conduit;
directing a propellant into the conduit, directing a flush into the conduit; and
removing the conduit from the animal's mouth.
